(19) United States
(12) Patent Application Publication
(10) Pub. No.: US 2026/0306609 A1
(43) Pub. Date: Oct. 8, 2026

(54) TREATING A TYPE I INTERFERON UPREGULATION OR ABERRANT IL-33 UPREGULATION DISEASE OR CONDITION WITH POLYOMAVIRUS

(71) Applicant: THE GENERAL HOSPITAL CORPORATION, Boston, MA (US)

(72) Inventors: Shadmehr Demehri, Charlestown, MA (US); Yun Xia, Malden, MA (US)

(21) Appl. No.: 19/485,099

(22) PCT Filed: May 13, 2024
(86) PCT No.: PCT/US2024/029124

§ 371 Date: Nov. 17, 2025

Related U.S. Application Data
(60) Provisional application No. 63/502,935, filed on May 18, 2023.

(51) Int. Cl. A61K 39/12; A61K 9/00; A61K 39/00; A61K 45/06; A61P 37/06; C12N 7/00; C12N 15/86

(52) U.S. Cl. CPC A61K 39/12; A61K 9/0019; A61K 45/06; A61P 37/06; C12N 7/00; C12N 15/86; A61K 2039/5252; A61K 2039/54; C12N 2710/22034; C12N 2750/14143

(57) ABSTRACT

Provided herein are methods that use life or life-attenuated polyomavirus or an antigenic protein, protein fragment, or peptide thereof (e.g., HPyV6, MCPyV, and/or HPyV7) to block the type I interferon pathway and IL-33 induction, thereby protecting against autoimmune and inflammatory diseases, such as, but not limited to systemic lupus erythematosus.

### CLAIM OF PRIORITY

This application claims the benefit of U.SA. Provisional Application Ser. No. 63/502,935, filed on May 18, 2023. The entire contents of the foregoing are incorporated herein by reference.

### TECHNICAL FIELD

The present disclosure relates to methods of using polyomavirus for the treatment of a type I interferon upregulation or aberrant IL-33 upregulation disease or condition (e.g., systemic lupus erythematosus (SLE)).

### BACKGROUND

Systemic lupus erythematosus (SLE) is a systemic autoimmune disease in which the immune system attacks the patient's own tissues; however, the mechanism of the aberrant immune response in this disease remains unclear [1].

Several predisposing factors have been implicated in the etiology of SLE, which include contributions from the environment, stochastic factors, and genetic susceptibility. Viral infection has been proposed as a potential contributor to SLE and Epstein-Barr virus and cytomegalovirus as SLE triggers have been described [2]. On the other hand, Helicobacter pylori, hepatitis B virus, lactate dehydrogenase virus, and parasite infections are suggested to play a protective role in lupus [3, 4].

### SUMMARY

Provided herein are methods of treating a condition associated with type 1 interferon overexpression in a patient comprising administering a therapeutically effective amount of a pharmaceutical composition comprising a polyomavirus to the patient.

In some embodiments, the polyomavirus is a live polyomavirus. In some embodiments, the polyomavirus is a live attenuated polyomavirus. In some embodiments, the polyomavirus is selected from HPyV6 and HPyV7. In some embodiments, the polyomavirus is administered by intradermal injection. In some embodiments, the condition is lupus.

Provided herein are methods for treating systemic lupus erythematosus, rheumatoid arthritis, Aicardi-Goutières syndrome, Dermatomyositis, systemic sclerosis, or Sjogren's syndrome, in a subject, the method comprising administering a therapeutically effective amount of live or live-attenuated polyomavirus, or an antigenic protein, protein fragment, or peptide thereof, to a subject identified as having lupus.

Provided herein are methods of vaccinating a subject against systemic lupus erythematosus, rheumatoid arthritis, Aicardi-Goutières syndrome, Dermatomyositis, systemic sclerosis, or Sjogren's syndrome, or reducing the risk of developing systemic lupus erythematosus, rheumatoid arthritis, Aicardi-Goutières syndrome, Dermatomyositis, systemic sclerosis, or Sjogren's syndrome, in a subject, the method comprising administering a therapeutically effective amount of live or live-attenuated polyomavirus, or antigenic protein, protein fragment, or peptide thereof, to the subject in need thereof.

In some embodiments, the live or live-attenuated polyomavirus, or antigenic protein, protein fragment, or peptide thereof is selected from MPyV, HPyV6 or HPyV7. In some embodiments, the polyomavirus is a live polyomavirus. In some embodiments, the polyomavirus is a live-attenuated polyomavirus. In some embodiments, the polyomavirus is delivered using a vector. In some embodiments, the vector is a viral vector. In some embodiments, the viral vector is an adeno-associated virus (AAV) vector. In some embodiments, the subject is a mammal. In some embodiments, the subject is a human. In some embodiments, the method is used to treat systemic lupus erythematosus (SLE). In some embodiments, the live or live-attenuated polyomavirus, or an antigenic protein, protein fragment, or peptide thereof are administered systemically. In some embodiments, the systemic administration comprises intradermal, subcutaneous, or intraperitoneal injections.

The method of any of the above claims, wherein treatment is monitored by observed dermatitis, size of spleen and/or lymph nodes, lupus nephritis, anti-DS DNA antibodies in the serum, urine protein concentration, or inflammatory cytokines in the skin. The method of any of the above claims, further comprising administering to the subject an additional therapy for lupus.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein, including but without limitation, PCT Patent Publications WO2022/112720 and WO2022/159652, are hereby incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 MPyV infection does not induce the expression of type I interferon-stimulated genes (ISGs). ISGs expression in Pam212 mouse keratinocytes infected with MPyV at a multiplicity of infection (MOI) of 20. Gene expression is compared with the control group at each time point post-infection. Viral major capsid protein gene (VP1) and large T-antigen (LTag) RNA expression are shown to confirm the efficacy of viral infection.

FIG. 2 MPyV infection does not upregulate MHC-I expression on the infected cells. MHC-I expression on Pam212 mouse keratinocytes 24 hours after infected with MPyV at MOI of 20.

FIG. 3 MPyV infection blocks the induction of the type I interferon-stimulated gene by type I interferon stimulators. Mx/expression in Pam212 mouse keratinocytes, mouse embryonic fibroblasts (MEF) and HC11 mouse breast epithelial cells infected with MPyV at MOI of 20. Three days (Pam212 (FIG. 3B) and HC11 (FIG. 3A)) or one day (MEF (FIG. 3C)) after infection, cells are treated with ds-DNA, DMXAA, poly(I:C) or carrier control. 9 hours later, Mx/expression is compared with the control group.

FIG. 4 MPyV infection blocks the induction of type I interferon-stimulated genes by TLR agonists. ISGs expression in MEFs infected with MPyV at MOI of 20. One day after infection, cells are treated with LPS, imiquimod or carrier control. 9 hours later, Mx1 and IL6 expressions are compared with the control group.

FIG. 5 MPyV infection blocks the induction of Il33 expression by poly(I:C). IL-33 expression in Pam212 keratinocytes infected with MPyV or virus-like particles (VLP) at MOI of 20. Three days after infection, cells are treated with poly(I:C) or carrier control. 9 hours later, IL-33 expression is compared with the control group.

FIG. 6 MPyV persists in mouse skin after intradermal infection. MPyV LTag RNA measured in WT mouse epidermis and dermis up to 12 weeks after infection with 2×10⁶ plaque-forming unit (PFU) of live MPyV. Viral LTag RNA level is measured relative to mouse Gapdh expression.

FIG. 7 MPyV is detectable in several vital organs after intradermal infection. MPyV LTag RNA measured in WT mouse organs at 3 weeks post-infection with 2×10⁶ PFU of live MPyV. Viral LTag RNA level is measured relative to mouse Gapdh expression.

FIG. 8 MPyV infection ameliorates the cutaneous manifestations of UVB-induced SLE in MRL/lpr mice. A schematic diagram of MPyV infection and UVB treatment in MRL/lpr mice. Skin dermatitis in 16-week-old MRL/lpr mice after receiving 2×10⁶ PFU of live MPyV versus virus-like particles (VLP) intradermally and being treated with 300 mJ/cm² UVB three times per week for 8 weeks.

FIG. 9 MPyV infection suppresses systemic manifestations of UVB-induced SLE in MRL/lpr mice. Spleen and lymph node sizes in 20-week-old MRL/lpr mice after receiving 2×10⁶ PFU of live MPyV versus VLP intradermally and being treated with 300 mJ/cm² UVB three times per week for 10 weeks.

FIG. 10 MPyV infection blocks glomerulonephritis in UVB-treated MRL/lpr mice. Representative H&E-stained images of kidneys in 20-week-old MRL/lpr mice after receiving 2×10⁶ PFU of live MPyV versus VLP intradermally and being treated with 300 mJ/cm² UVB three times per week for 10 weeks. Arrows point to the inflammation surrounding the glomeruli in the VLP-treated MRL/lpr kidney.

FIG. 11 MPyV infection suppresses SLE in UVB-treated MRL/lpr mice. Serum anti-dsDNA in MRL/lpr mice after receiving 2×10⁶ PFU of live MPyV versus VLP intradermally and being treated with 300 mJ/cm² UVB three times per week (two-way ANOVA).

FIG. 12 MPyV infection prevents proteinuria in UVB-treated MRL/lpr mice. Urine protein levels in MRL/lpr mice after receiving 2×10⁶ PFU of live MPyV versus VLP intradermally and being treated with 300 mJ/cm² UVB three times per week (two-way ANOVA).

FIG. 13 MPyV infection blocks the induction of ISGs in UVB-treated MRL/lpr mice. ISGs expression in the organs (spleen, top; kidney, middle; and skin, bottom) of 20-week-old MRL/lpr mice after receiving 2×10⁶ PFUof live MPyV versus VLP intradermally and being treated with 300 mJ/cm² UVB three times per week for 10 weeks.

FIG. 14 HPyV6 infection significantly suppresses expression interferon-stimulated genes (ISGs) in fibroblasts compared to virus-like particles (VLP). FIG. 14A: HPyV6 large T antigen was expressed at high levels after HPyV6 virion colonizing in human embryonic fibroblasts. FIG. 14B: Expression of the interferon-stimulated gene (ISG), IFN-ß, in fibroblasts infected with either HPyV6 or VLP. FIG. 14C: Expression of interferon-stimulated gene (ISG), MX1, in fibroblasts infected with either HPyV6 or VLP. FIG. 14D: Expression of interferon-stimulated gene (ISG), ISG15, in fibroblasts infected with either HPyV6 or VLP. Cells were stimulated by ds-DNA (light grey circles) or poly(I:C) (dark grey circles).

FIG. 15 MPyV 315YF preparation suppresses expression of interferon-stimulated genes (ISGs) in mouse embryonic fibroblasts (MEFs) compared to virus-like particles (VLP). FIG. 15A: Schematic of the MPyV genome containing plasmid A2R1-pBluescript as the backbone and the 315YF mutant virus genome. FIG. 15B: Expression of interferon-stimulated gene (ISG), MX1, in mouse embryonic fibroblasts (MEFs) infected with either MPyV 315YF or VLP. FIG. 15C: Expression of interferon-stimulated gene (ISG), ISG15, in MEFs infected with either MPyV 315YF or VLP. FIG. 15D: Expression of interferon-stimulated gene (ISG), OAS2, in MEFs infected with either MPyV 315YF or VLP. Cells were stimulated by 1 μg/mL ds-DNA (light grey circles) or 1 μg/mL poly(I:C) (dark grey circles).

FIG. 16 Live MPyV intradermal injection serves as a therapeutic strategy against lupus in MRL/lpr mice. FIG. 16A: schematic diagram of MPyV infection and UVB treatment in MRL/lpr mice. FIG. 16B: Skin dermatitis in 16-week-old MRL/lpr mice after receiving 2×10⁶ PFU of live MPyV versus virus-like particles (VLP) intradermally and being treated with 300 mJ/cm² UVB thrice weekly for 8 weeks.

FIG. 16C: Serum anti-dsDNA antibody in MRL/lpr mice at 16 weeks of age (n=10 per group). FIG. 16D: Urine albumin concentrations in MRL/lpr mice at 16 weeks of age (n=10 per group). Unpaired Mann-Whitney U test was used for analysis (C and D).

### DETAILED DESCRIPTION

Type I interferons have been proved to play an important role in immune dysregulation in lupus-prone mice and human lupus patients [19]. Studies have found that patients treated with IFN-a for cancer and hepatitis therapy could develop a lupus-like disease with autoantibodies in serum. These findings suggest that type I interferon signaling may be sufficient to break the immune tolerance and induce autoimmune diseases [20]. 50%-75% of adults and about 90% of children with lupus show an increased expression of type I interferon-stimulated genes [21]. Considering the importance of type I interferon in the pathogenesis of lupus, many strategies have been tested to downregulate the interferon pathway in patients with lupus. However, it has been difficult to develop an effective strategy for long-term suppression of the aberrantly active type I interferon in lupus and other autoimmune/inflammatory diseases.

Merkel Cell Polyomavirus (MCPyV) positive cancer cell lines cannot respond to dsDNA and DMXAA stimulation to produce type I interferons [22]. Other studies also have found that overexpression of SV40 large T antigen, a polyomavirus that is found in both monkeys and humans, also results in reduced interferon response to DNA sensor agonists [23]. The type I interferon pathway suppression by polyomavirus presented here is a novel strategy to prevent and treat lupus and other autoimmune/inflammatory diseases associated with active type I interferon signaling. Interestingly, it has been reported that patients with autoimmune/inflammatory skin diseases have reduced overall polyomaviruses load in their skin compared with healthy individuals [24]. Presented herein are methods demonstrating favorable effect of polyomavirus colonization against autoimmune disease in a host that offer new insights about the beneficial impact of commensal viruses against autoimmune/inflammatory diseases and reveal a therapeutic strategy to combat these common diseases.

### Conditions

The methods described herein use polyomavirus to block the type I interferon pathway and IL-33 induction and protects against autoimmune and inflammatory diseases. Thus, live and live-attenuated human polyomaviruses (e.g., HPyV6, MCPyV, and HPyV7) can serve as novel therapeutic agents for the treatment and prevention of autoimmunity, inflammatory diseases, fibrosis, and cancer associated with type I interferon and/or IL-33 overexpression.

The conditions that can be treated using the methods of the disclosure include diseases associated with aberrant type I interferon upregulation, such as autoimmune diseases. In some embodiments, conditions treated include those with high level of type I interferon in the serum of patients with systemic lupus erythematosus (SLE) as well as other systemic autoimmune diseases, including rheumatoid arthritis, Aicardi-Goutières syndrome, Dermatomyositis, systemic sclerosis and Sjogren's syndrome.

In rheumatoid arthritis, blocking type I interferon reduces both the interferon signature and the development of arthritis [26]. Systemic lupus erythematosus is a prototypic type I interferon-mediated autoimmune disease [27]. Aicardi-Goutières syndrome (AGS) is highlighted by a fundamental link between nucleic acid metabolism, innate immune sensors and type I interferon induction [28]. In Dermatomyositis (DM), serum interferon-α activity is increased and is associated with high serum muscle-derived enzymes. Janus kinase (JAK) inhibitors, ruxolitinib and tofacitinib, have been reported to be effective in the treatment of DM [29]. In Sjögren Syndrome (SjS), interferon-α expression levels at the transcript or protein levels are found to be increased in the labial salivary glands and blood, and a positive correlation is identified between genes induced by type I interferons and titers of anti-Ro/SSA, anti-La/SSB autoantibodies, European League Against Rheumatism (EULAR) SjS disease activity index, biological markers of activity, and B-cell activating factor (BAFF) mRNA in the patients' monocytes [30]. In Systemic Sclerosis, elevated levels of type I interferons are associated with severe symptoms in the skin, lung, and skeletal muscle, and the disease activity can be suppressed after treatment with the anti-interferon-α/β receptor (IFNAR) antibody, anifrolumab [31]. Additionally, the methods of the disclosure can be used to treat subjects with viral infections. Type I interferons can be harmful in virus infection by triggering inflammation and tissue damage that exacerbate the disease. They can cause morbidity and mortality during influenza virus infection [32]. A mice SARS-COV-2 model shows inhibition of type I interferon responses reduces severe lung inflammation induced by SARS-COV-2 [33]. Thus, the methods can be used to reduce deleterious inflammatory responses associated with viral infections. The invention also finds use in treating subjects with bacterial infections. Interferon-α/β receptor (IFNAR)-deficient mice are more resistant to infection with Francisella tularensis and Listeria monocytogenes[35 ] than are WT mice. M. tuberculosis -infected IFNAR knockout mice have decreased and late mortality compared with WT animals [36].

Additionally, the methods of the disclosure can be used to treat endotoxin shock. Type I interferon deficiency reduces LPS [37] and colon ascendens stent [38] induced endotoxin shock and lethality.

Additionally, the method of the disclosure can be used to treat diseases associated with aberrant IL-33 upregulation. Such diseases include cancer, including those that develop in the context of chronic inflammation, allergic, fibrotic, infectious, or chronic inflammatory disease, chronic inflammatory conditions include asthma, atopic dermatitis, allergic contact dermatitis, hepatitis, pancreatitis, or inflammatory bowel disease (IBD), fibrotic disorders including organ fibrosis and systemic sclerosis, and acute coronary syndrome or coronary artery disease (CAD).

### Subjects

The methods described herein can be used to treat any of the type I interferon upregulation and aberrant IL-33 upregulation diseases or conditions described herein. In some embodiments, the subject has an increased risk of developing a type I interferon upregulation or aberrant IL-33 upregulation disease or condition described herein. In some embodiments, the subject has a type I interferon upregulation or aberrant IL-33 upregulation disease or condition described herein. In some embodiments, the subject is a mammal (e.g., a human or a non-human veterinary subject, e.g., a cat, dog, cow, horse, pig, mouse, rat, or rabbit).

### Polyomavirus

Polyomaviruses are small, nonenveloped double-stranded DNA viruses that are widespread in humans. In immunocompetent hosts, these viruses colonize the tissues after primary infection, which appear to cause little or no symptoms during the lifespan of the immunocompetent host. A high seroprevalence of polyomaviruses was observed in healthy adult blood donors, and it was found that the seroprevalence of simian vacuolating virus 40 (SV40) was 9%, human polyomavirus 6 (HPyV6) 88.2%, human polyomavirus 7 (HPyV7) 65.7.2%, BK polyomavirus (BKPyV) 82%, JC polyomavirus (JCPyV) 39%, primate lymphotropic polyomavirus (LPyV) 15%, KI poly omavirus (KIPyV) 55%, WU polyomavirus (WUPyV) 69% and Merkel Cell Polyomavirus (MCPyV) 65%. Polyomaviruses DNA is detectable in healthy human blood: MCPyV 3.8%, JCPyV and TS polyomavirus (TSPyV) DNA 0.5%, and HPyV9 0.4%. BKPyV, WUPyV, HPyV6, MWPyV, and LIPyV DNA were detected at 0.1%-0.2% blood samples. Human MCPyV, HPyV6, and HPyV7 are considered as cutaneous viruses. MCPyV were found at 40-80% DNA positive in healthy individuals. HPyV6 and HPyV7 were found in 14-50% and 11% respectively in normal human skin. Previous studies have shown high polyomavirus seroprevalence and frequent presence of polyomavirus-specific T cells in healthy adults. However, in the elderly population, Merkel cell polyomavirus (MCPyV) integration into host genome is the primary oncogenic event in ~80% of Merkel cell carcinoma (MCC) cases while the remaining virus-negative MCC cases are associated with ultraviolet (UV) radiation. MCPyV-induced MCCs are highly immunogenic and are associated with prominent intratumoral T and natural killer (NK) cell infiltrates.

The present disclosure is inclusive of polyomaviruses that can establish persistent infection in humans. The Polyomavirus genome is characterized by a specific organizational structure, featuring a double-stranded DNA (dsDNA) genome containing distinct early and late regions. The early region encodes the T antigens, while the late region encodes structural viral proteins, both situated on opposite strands. These regions are demarcated by a noncoding control region. The genetic distance observed in the large T antigen (LTAg) coding sequence to members of the most closely related species exceeds 15%. When two polyomaviruses exhibit a genetic distance of less than 15%, considerations of biological properties such as host specificity, disease association, tissue tropism, etc., may warrant the establishment of a new species. Provided herein are the genome sequences of select human poly omaviruses: HPyV6 (Accession No. NC_014406), HPyV7 (Accession No. NC_014407), and MCPyV (Accession No. NC_010277).

### Pharmaceutical Compositions

The polyomaviruses of the disclosed method can be formulated in a pharmaceutical composition. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include parenteral, e.g., transdermal, intradermal, subcutaneous, intramuscular, or subcutaneous administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, transdermal, intradermal, intramuscular, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, administering a therapeutically effective amount of live or live-attenuated commensal polyomavirus, or antigenic protein, protein fragment, or peptide thereof, to a subject identified as having a type I interferon upregulation or aberrant IL-33 upregulation disease or condition. Systemic administration of a pharmaceutical or therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

### Method of Treatment

Provided herein are methods for treating a type I interferon upregulation or aberrant IL-33 upregulation disease or condition in a subject in need thereof, the method comprising administering a therapeutically effective amount of live or live-attenuated commensal polyomavirus, or antigenic protein, protein fragment, or peptide thereof, to a subject identified as having a type I interferon upregulation or aberrant IL-33 upregulation disease or condition (e.g., SLE).

The methods include administering a therapeutically effective amount of live or live-attenuated commensal polyomavirus, or antigenic protein, protein fragment, or peptide thereof, to a subject identified as having a type I interferon upregulation or aberrant IL-33 upregulation disease or condition (e.g., any of the a type I interferon upregulation or aberrant IL-33 upregulation disease or condition identified herein). In some embodiments, the live or live-attenuated commensal polyomavirus, or antigenic protein, protein fragment, or peptide thereof, is selected from MPyV, MCPyV, HPyV7, HPyV6, BKPyV, JCPyV, LPyV, KIPyV, WUPyV, and/or TSPyV polyomavirus strains and/or MPyV, MCPyV, HPyV7, HPyV6, BKPyV, JCPyV, LPyV, KIPyV, WUPyV, and/or TSPyV antigenic proteins, protein fragments, or peptides. In some embodiments, subject is administered two or more live or live-attenuated commensal polyomaviruses, or antigenic proteins, protein fragments, or peptides thereof, selected from MPyV, MCPyV, HPyV7, HPyV6, BKPyV, JCPyV, LPyV, KIPyV, WUPyV, and/or TSPyV polyomavirus strains and/or MPyV, MCPyV, HPyV7, HPyV6, BKPyV, JCPyV, LPyV, KIPyV, WUPyV, and/or TSPyV antigenic proteins, protein fragments, or peptides. In some cases, the antigenic protein, protein fragment, or peptide can be expressed by a vector (e.g., a viral vector, a non-viral vector, a DNA vector, or an RNA vector). For example, the viral vector can be a lentivirus, an adenovirus, an adeno-associated virus, a retrovirus, or a herpes simplex virus. The non-viral vector can be a liposome, an exosome, an extracellular vesicle, a polymer, a nanoparticle, a peptide, or a dendrimer.

The methods described herein can be used to treat a type I interferon upregulation or aberrant IL-33 upregulation disease or condition in a subject in need thereof. In some embodiments, the subjects have an increased risk of developing a type I interferon upregulation or aberrant IL-33 upregulation disease or condition. For example, the subject may have a family history and/or personal history of an autoimmune condition (e.g., lupus). In some embodiments, subjects are mammals.

Methods for treating a mammal having a type I interferon upregulation or aberrant IL-33 upregulation disease or condition (e.g., SLE) can include identifying the mammal as having type I interferon upregulation or aberrant IL-33 upregulation. Examples of methods for identifying the mammal as having type I interferon upregulation or aberrant IL-33 upregulation include, without limitation, physical examination, laboratory tests (e.g., blood or urine), biopsy, endoscopy, genetic tests, or other methods of identifying type I interferon upregulation or aberrant IL-33 upregulation as known in the art.

The methods can also include administration of one or more other treatments known in the art for type I interferon upregulation or aberrant IL-33 upregulation (e.g., inflammation and/or autoimmune conditions). For example, a combination treatment with the methods described herein can include anti-inflammatory drugs, corticosteroids, pain-killing medication, immunosuppressant drugs, or treatment for deficiencies. Examples of anti-inflammatory drugs include, without limitation, diclofenac, naproxen, flurbiprofen, celecoxib, fenoprofen, tolmetin, diflunisal, ibuprofen, etodolac, nabumetone, ketoprofen, mefenamic acid, ketorolac, magnesium salicylate, indomethacin, aspirin, piroicam, sulindac, meloxicam, or oxaprozin. Examples of corticosteroids include, without limitation, methylprednisolone, betamethasone, budesonide, decadron, pulmicort, entocort EC, dexamethasone, hydrocortisone, alclometasone, dexamethasone intensol, beclomethasone, prednisolone, triamcinolone, cortisone acetate, fluticasone propionate, or clobetasol. Examples of pain-killing medication include, without limitation, morphine, diclofenac, aspirin, meperidine, acetaminophen, piroxicam, oxycodone, hydrocodone, tramadol, ketorolac, indomethacin, naproxen, ibuprofen, hydromorphone, or methadone. Examples of immunosuppressant drugs include, without limitation, mycophenolate mofetil, cyclosporine, everolimus, abatacept, adalimumab, anakinra, certolizumab, entercept, infliximab, ixekizumab, natalizumab, rituximab, secukinumab, tocilizumab, ustekinumab, vedolizumab, golimumab, ATGAM, ASTAGRAF XL, mycophenolic acid, azathioprine, sirolimus, leflunomide, ciclosporin, methotrexate, muromonab, basiliximab, prednisone, tacrolimus, omalizumab, daclizumab, alemtuzumab, or fingolimod.

In cases where a live or live-attenuated commensal polyomavirus, or antigenic protein, protein fragment, or peptide thereof provided herein and one or more additional type I interferon upregulation or aberrant IL-33 upregulation treatments are provided separately, the administration of a live or live-attenuated commensal polyomavirus, or antigenic protein, protein fragment, or peptide thereof provided herein can be in any order relative to the administration of one or more additional type I interferon upregulation or aberrant IL-33 upregulation treatments. For example, a live or live-attenuated commensal polyomavirus, or antigenic protein, protein fragment, or peptide thereof provided herein can be administered to a mammal prior to, concurrent with, or following administration of one or more additional type I interferon upregulation or aberrant IL-33 upregulation treatments to the mammal.

Also provided herein are methods of vaccinating or reducing the risk of developing a type I interferon upregulation or aberrant IL-33 upregulation disease or condition (e.g., SLE) in a subject, the method comprising administering a therapeutically effective amount of live or live-attenuated commensal polyomavirus, or antigenic protein, protein fragment, or peptide thereof, to the subject in need thereof.

The live or live-attenuated commensal polyomavirus, or antigenic protein, protein fragment, or peptide thereof are administered in a therapeutically effective amount. A “therapeutically effective amount” is an amount sufficient to effect beneficial or desired results. For example, an effective amount is one that achieves a desired therapeutic effect, e.g., an amount necessary to treat a disease, or to reduce risk of development of disease or disease symptoms (also referred to as a prophylactically effective amount). An effective amount can be administered in one or more administrations, applications, or dosages. A therapeutically effective amount of a therapeutic compound (i.e., an effective dosage) depends on the therapeutic compounds selected. For example, a therapeutically effective amount of live or live-attenuated commensal polyomavirus, or antigenic protein, protein fragment, or peptide thereof provided herein can be effective to reduce the number of or severity of symptoms in a mammal (e.g., human). The live or live-attenuated commensal polyomavirus, or antigenic protein, protein fragment, or peptide thereof can be administered one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. Various factors can influence the actual amount used for a particular application. For example, the frequency of administration, duration of treatment, combination of other agents, site of administration, stage of disease (if present), and the anatomical configuration of the treated area may require an increase or decrease in the actual amount administered.

The frequency of administration of live or live-attenuated commensal polyomavirus (e.g., HPyV7, HPyV6, or MPyV) provided herein can be any frequency. For example, the frequency of administration can be from about four times a day to about once a month, or more specifically, from about twice a day to about once a week. In addition, the frequency of administration can remain constant or can be variable during the duration of treatment. As with the amount administered, various factors can influence the actual frequency of administration used for a particular application. For example, the amount (dose), duration of treatment, combination of agents, site of administration, stage of disease (if present), and the anatomical configuration of the treated area may require an increase or decrease in administration frequency. For example, the methods can include administering a first dose, followed by one or more additional doses at a later time (e.g., a “booster” dose), e.g., 1, 2, 3, 4, 5, 6, or 7 days from a previously administered dose. For example, administration can include administering two, three, four, or five doses, administered 1, 2, 3, 4, 5, 6, or 7 days apart. The length of time between multiple doses can be the same (e.g., 2 days between each dose, 3 days between each dose, 4 days between each dose, etc.), or the length of time between multiple doses can be different between each dose (e.g., 4 days between the first and second dose, 3 days between the second and third dose, and 2 days between the third and fourth dose).

Dosage, toxicity and therapeutic efficacy of the therapeutic compositions can be determined by standard pharmaceutical procedures in cell cultures or experimental animals, e.g., for determining the LD 50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit high therapeutic indices are preferred. While compositions that exhibit toxic side effects may be used, care should be taken to minimize and reduce side effects. The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compositions used in the methods described herein, the therapeutically effective dose can be estimated initially from cell culture assays. A dose may be formulated in animal models. Such information can be used to more accurately determine useful doses in humans.

### EXAMPLES

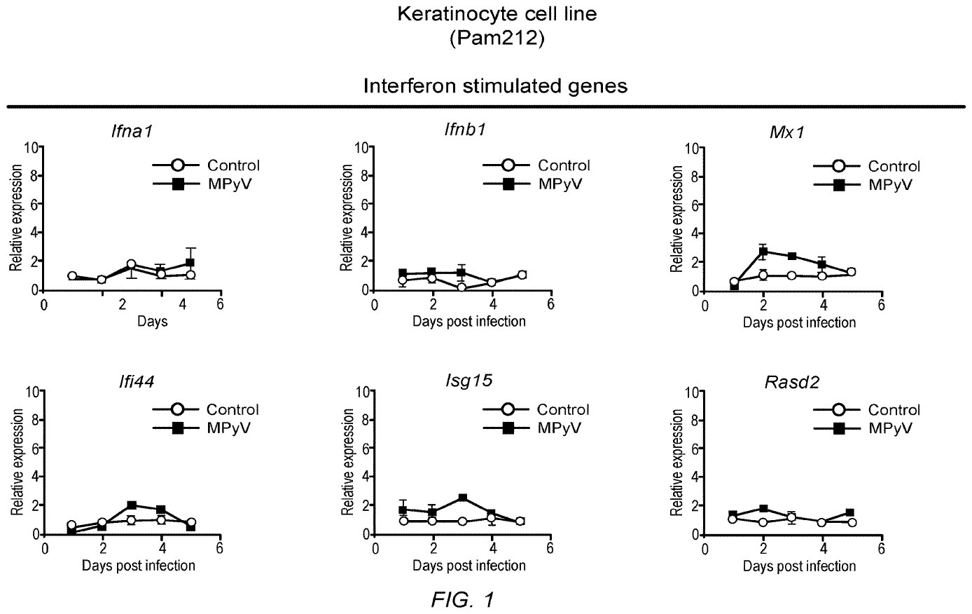

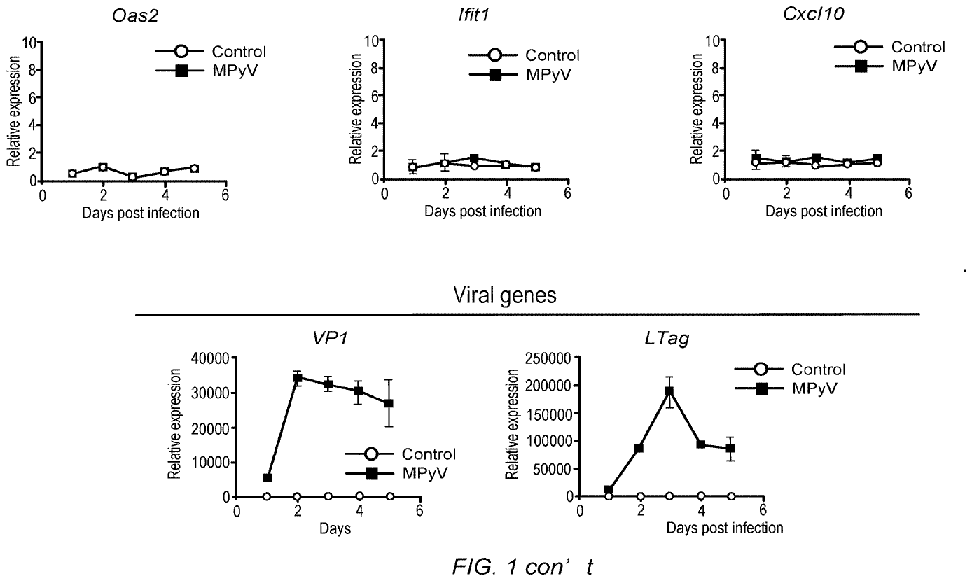

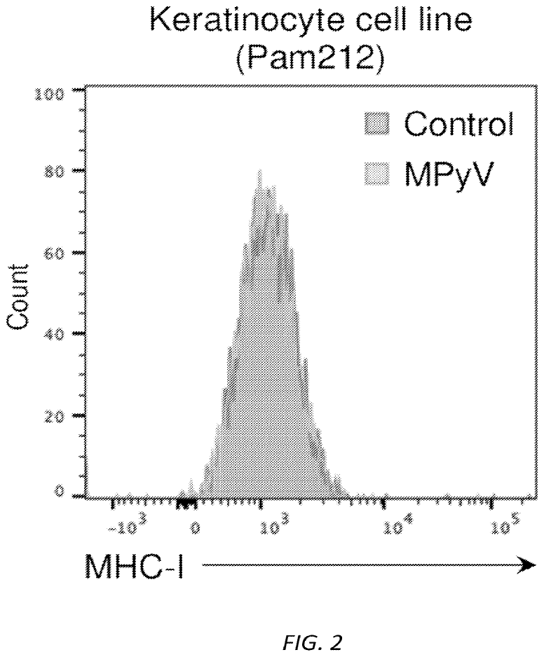

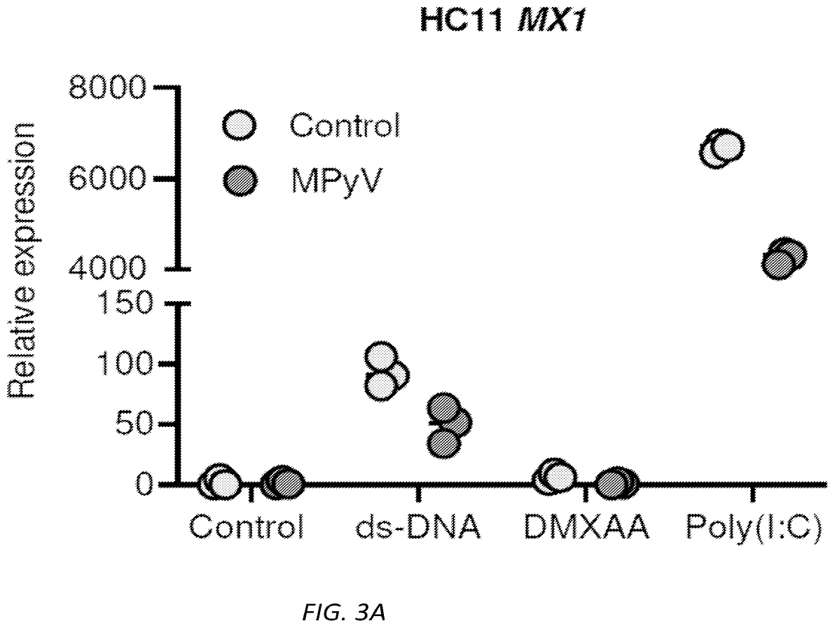

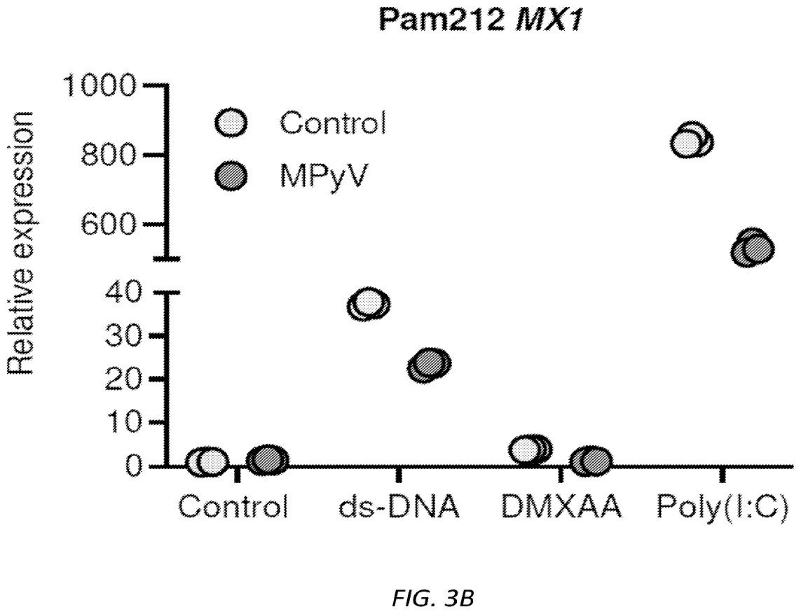

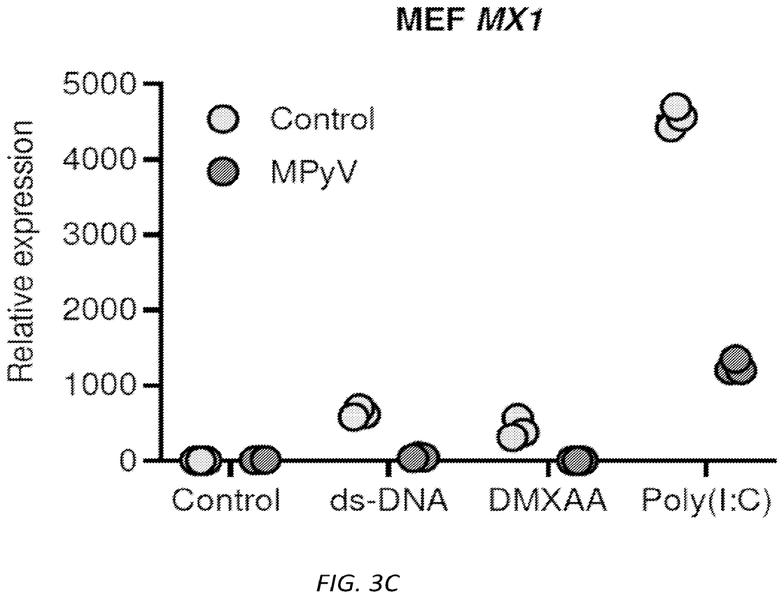

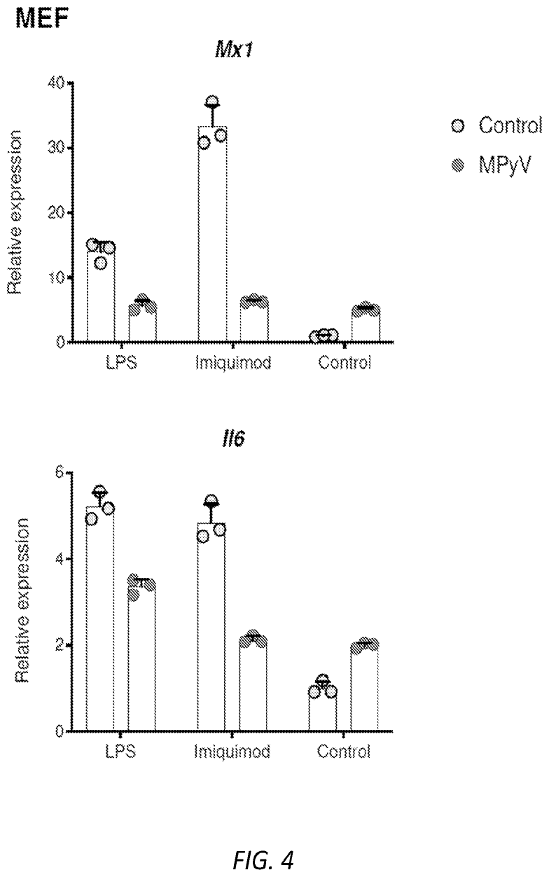

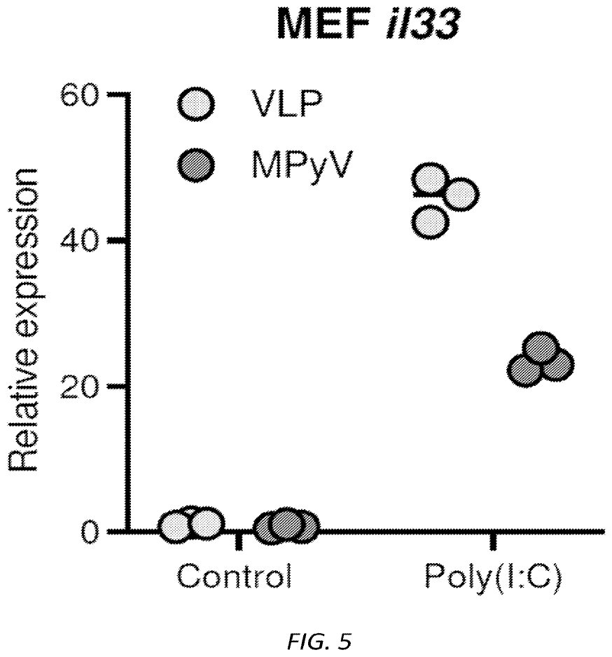

The invention is further described in the following examples, which do not limit the scope of the invention described in the claims. Shown herein, mouse polyomavirus (MPyV) infection did not induce the expression of type I interferon-stimulated genes (ISG) or major histocompatibility complex class I (MHC-I) in the infected cells (FIGS. 1 and 2). Surprisingly, MPyV inhibited type I interferon pathway activation by known activators of this pathway including double-stranded (ds)-DNA, DMXAA (STING agonist), poly(I:C) (TLR3 agonist), Lipopolysaccharides (LPS, TLR4 agonist) and imiquimod (TLR7/8 agonist) (FIGS. 3 and 4). In addition, MPyV infection suppressed IL-33 induction by poly(I:C) (FIG. 5).

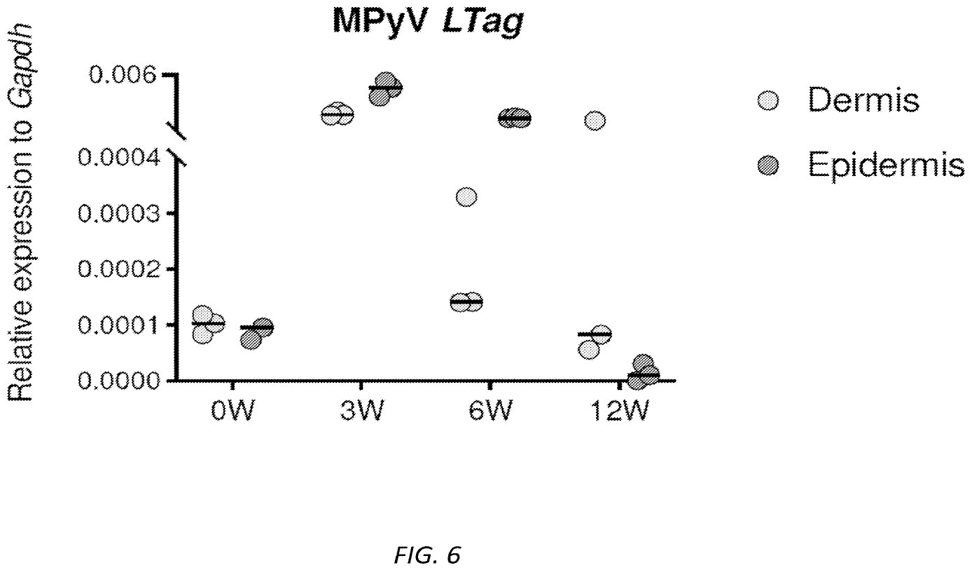

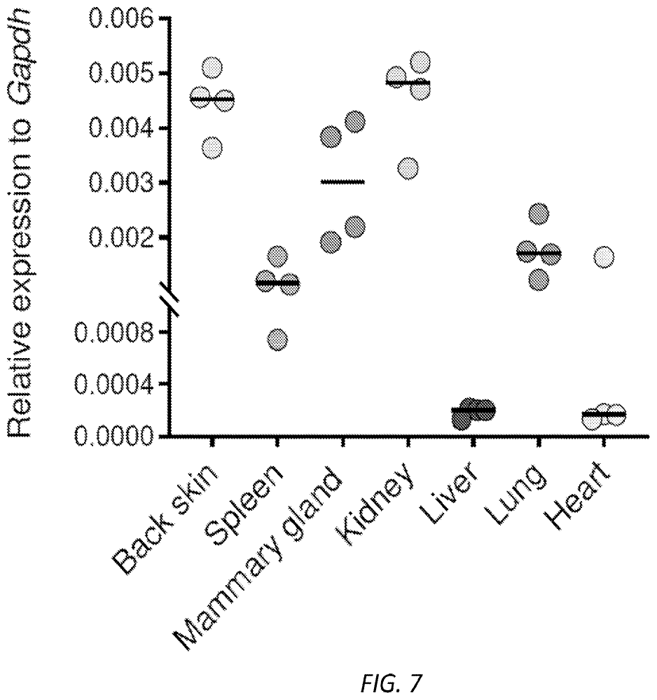

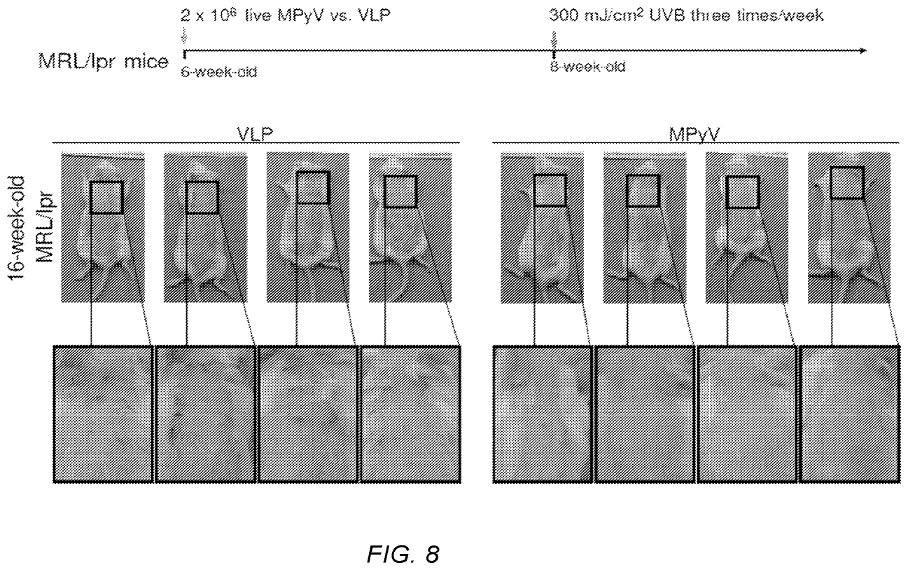

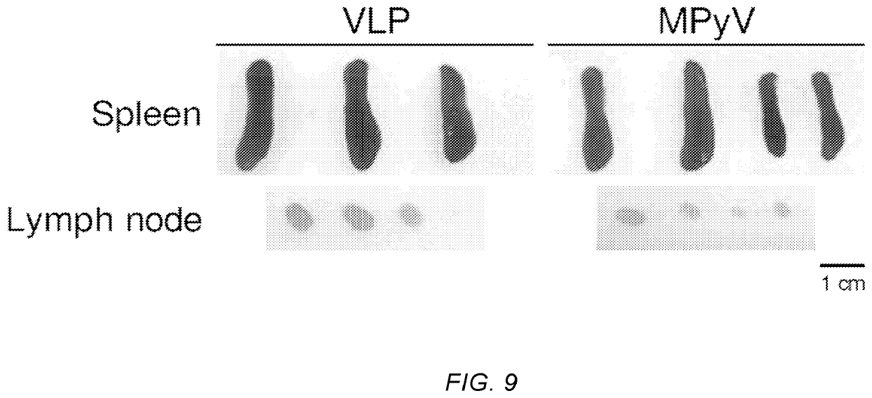

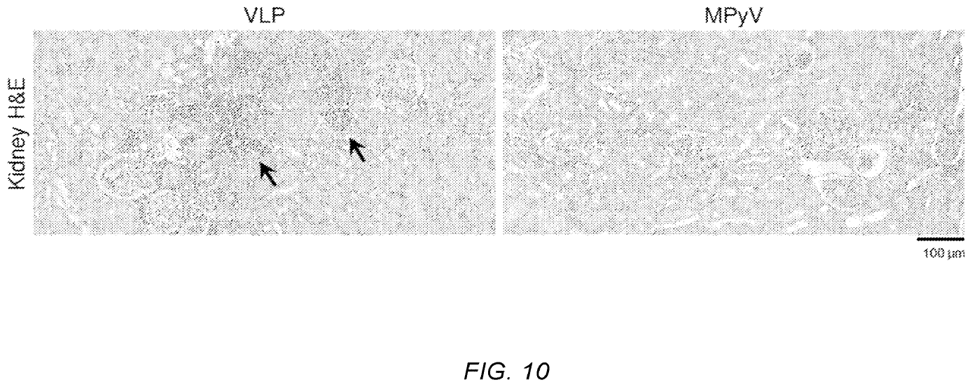

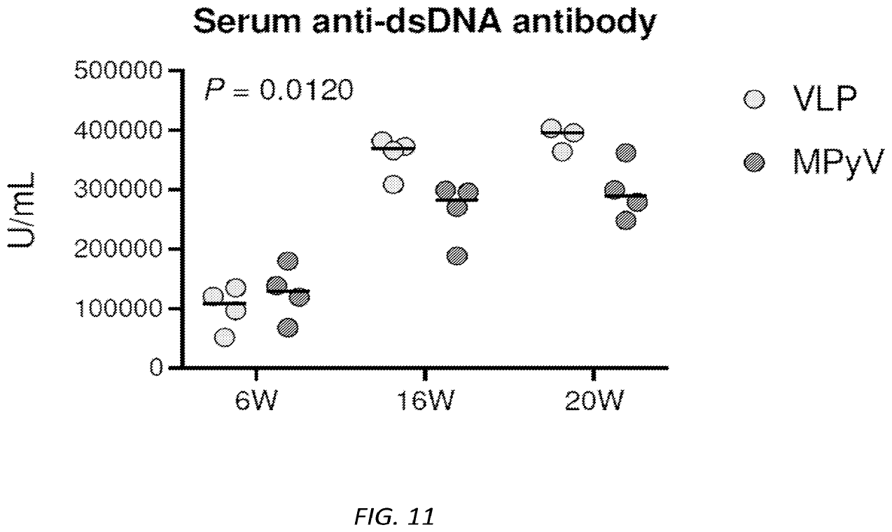

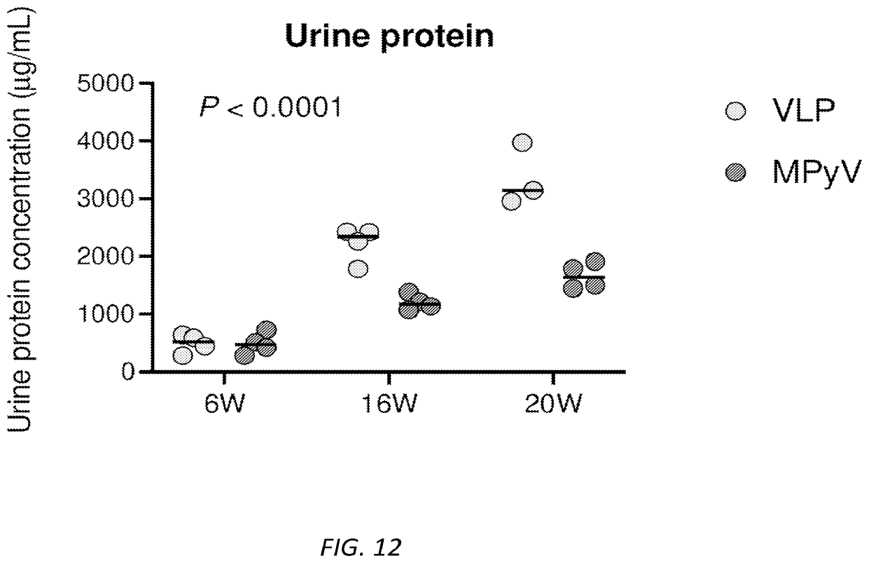

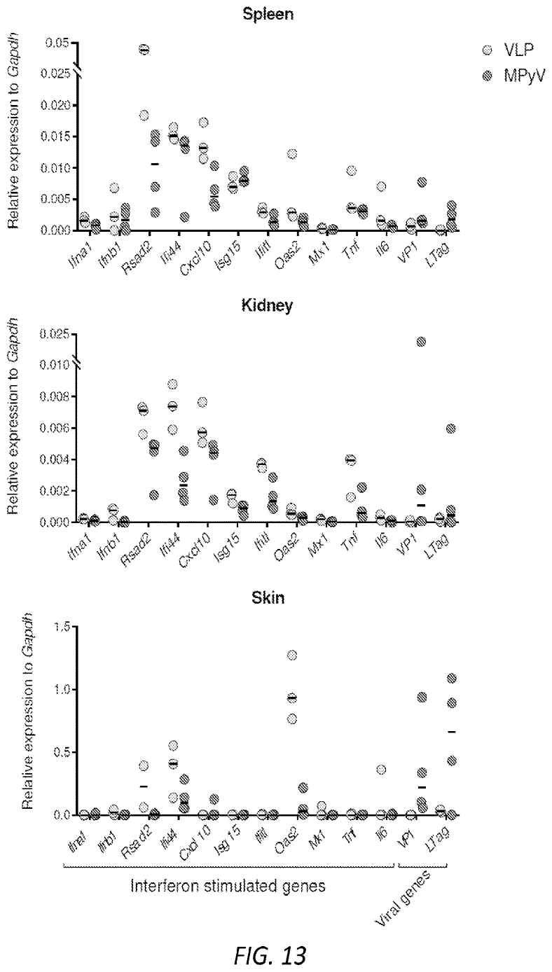

Additionally, whether MPyV blocked diseases that are associated with the aberrant activation of the type I interferon pathway was also investigated. In wild-type (WT) mice, intradermal injection of 2×10⁶ live MPyV led to persistent colonization of the animals in several vital organs for over 12 weeks post-infection with no sign of disease (FIGS. 6 and 7). In an MRL/lpr lupus mouse model, live MPyV infection suppressed lupus development compared with MPyV virus-like particle (VLP)-infected control group including reduced dermatitis, enlargement of mouse spleen and lymph node, lupus nephritis, anti-dsDNA antibodies in the serum, the protein concentration of the urine and the inflammatory cytokines in the spleen, kidney and skin (FIGS. 8 to 13).

### Example 1: HpyV6 Virion and VLP Preparation

Human polyomavirus 6 (HPyV6) virions were produced in vitro by transfecting 293TT cells with recombinant HPyV6 genomic DNA plasmids. The HPyV6 recombinant genome was prepared by first digesting 50 μg of plasmid pHPy V6-607a (Addgene #24727) with HindIII (Thermo Scientific, cat: FD0504) to release the cloned HPyV6 genome. The restriction enzyme was heat-inactivated, and the digested DNA was purified by gel DNA extraction kit, diluted to 5.5 μg/ml in 1×T4 DNA ligase buffer and treated with 12,000 U of T4 DNA ligase (NEB, cat: M 0202M) at 16 degrees overnight. The ligated HPyV6 genome DNA was then transfected into 293TT cells using Lipofectamine 2000, according to the manufacturer's instructions. Virions were harvested from the cells 7 days after transfection [14].

Human HPyV6 VLP control was produced by transfecting 293TT cells with recombinant p6VP1 plasmid expressing VP1 protein of HPyV6 (Addgene #24725). VLPs were harvested from the cells 7 days after transfection [14].

### Example 2: Human Embryonic Fibroblast Stimulation

3×10⁵ Human embryonic fibroblasts were seeded in 6 well plates and colonized with 250 Viral genome equivalent (VGE) per cell with HPyV6 virion and comparable VLP overnight. Twenty-four hours later, cells were stimulated with 1 μg/ml ds-DNA or 1 μg/ml poly(I:C) for another 9 hours. Cell RNA was purified with TRIzol and human IFN-β, MX1, and ISG15 were detected with qPCR.

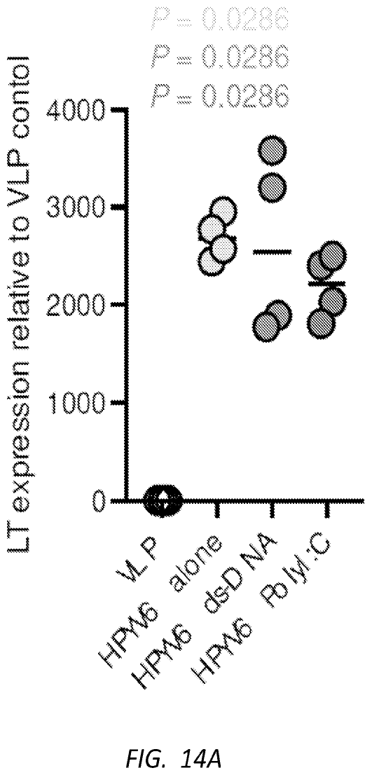

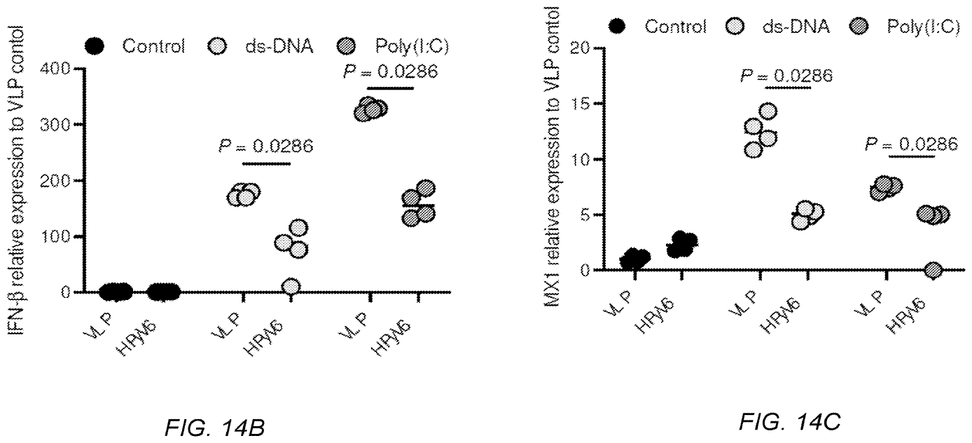

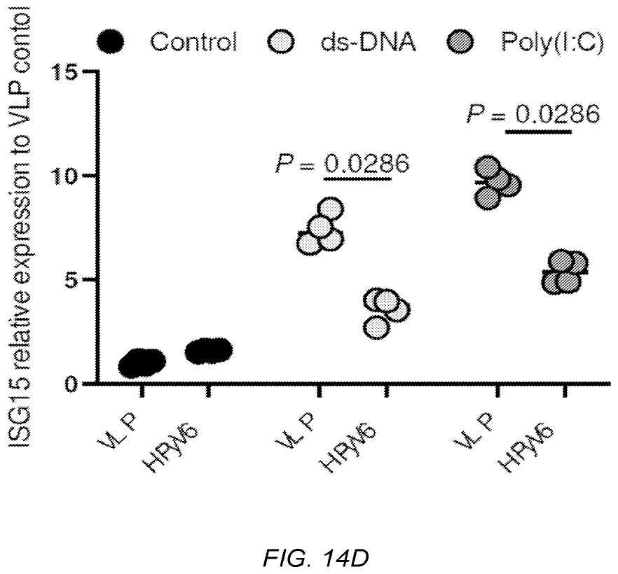

HPyV6 large T antigen was expressed at high levels after HPyV6 virion colonizing in human embryonic fibroblasts (FIG. 14A). The expression of interferon-stimulated genes (ISGs): IFN-β (FIG. 14B), MX1 (FIG. 14C), and ISG15 (FIG. 14D) that were induced by ds-DNA or poly(I:C) were significantly suppressed in HPyV6-infected compared with VLP-treated fibroblasts.

### Example 3: Attenuated Mouse Polyomavirus 315YF Preparation

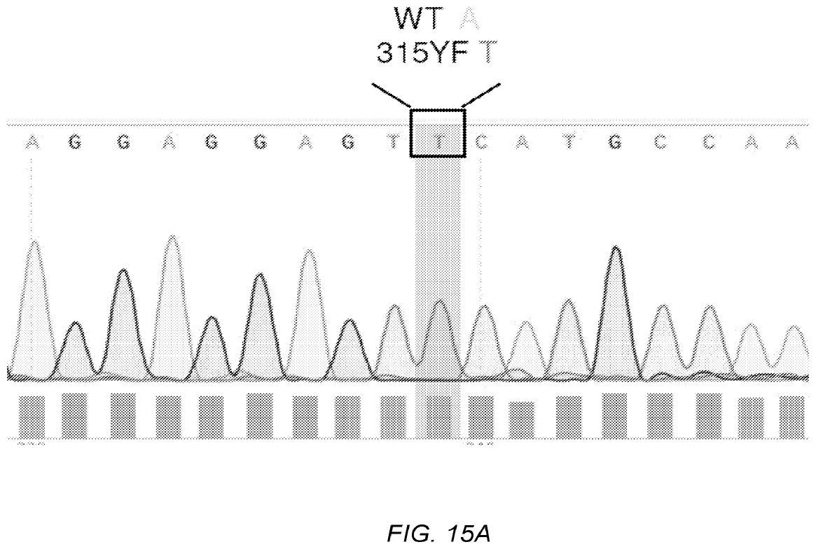

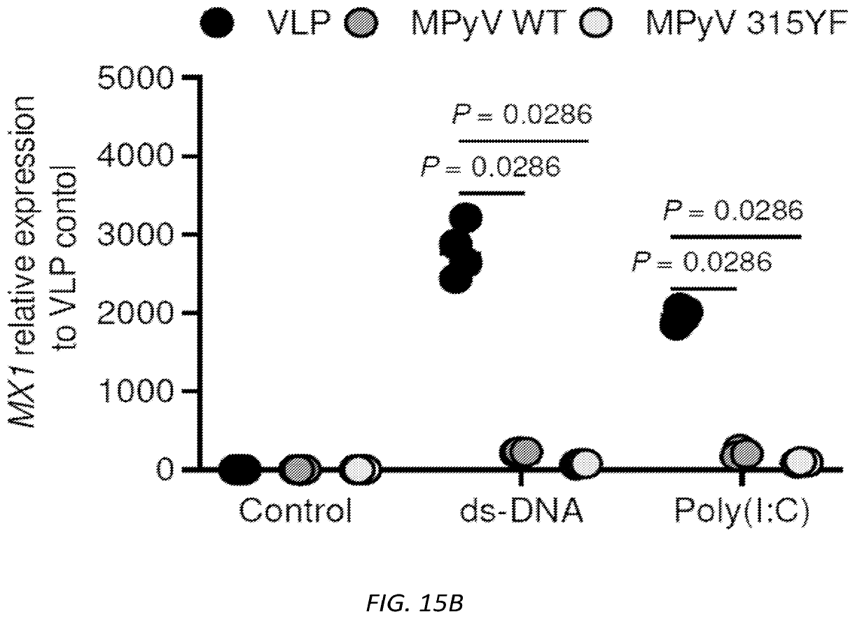

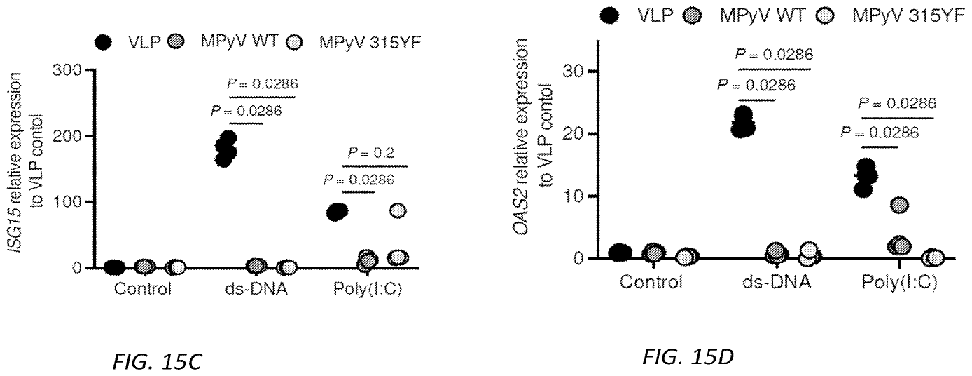

Middle T antigen in MPyV can be mitogenic and has the ability to transform normal fibroblasts in culture [15, 16]. The hydrophobic domain mutant form of MPyV (315YF strain) has been demonstrated to fail to induce cell transform [17, 18]. We used the MPyV genome containing plasmid A2R1-pBluescript as the backbone and generated the 315YF mutant virus genome (FIG. 15A). 50 μg A2R1-315YF-pBluescript prepared by first digesting with EcoRI (Thermo Scientific, cat: FD0274) for 1 hour. The restriction enzyme was heat-inactivated, and the digested DNA was purified by gel DNA extraction kit, diluted to 5.5 μg/ml in 1×T4 DNA ligase buffer, and treated with 12,000 U of T 4 DNA ligase (NEB, cat: M0202M) at 16 degrees overnight. The ligated MPyV 315YF genome DNA was then transfected into NMuMG cells using Lipofectamine 2000, according to the manufacturer's instructions. Viruses from NMuMG cell supernatants were harvested from the cells 7 days after transfection, virus titer was determined by plaque assay.

### Example 4: Mouse Embryonic Fibroblast (MEF) Attenuated MPyV 315YF Colonization and Stimulation

3×10⁵ MEFs were seeded in 6 well plates and colonized with 20 MOI WT and MPyV 315YF or comparable VLP overnight. Twenty-four hours later, cells were stimulated with 1 μg/ml ds-DNA or 1 μg/ml poly(I:C) for another 9 hours. Attenuated MPyV 315YF markedly suppressed the induction of MX1 (FIG. 15B), ISG15 (FIG. 15C) and OAS2 (FIG. 15D) expression by ds-DNA and poly(I:C).

### Example 5: Therapeutic Utilization of MPyV Suppressed Lupus in MRL/lpr Mice

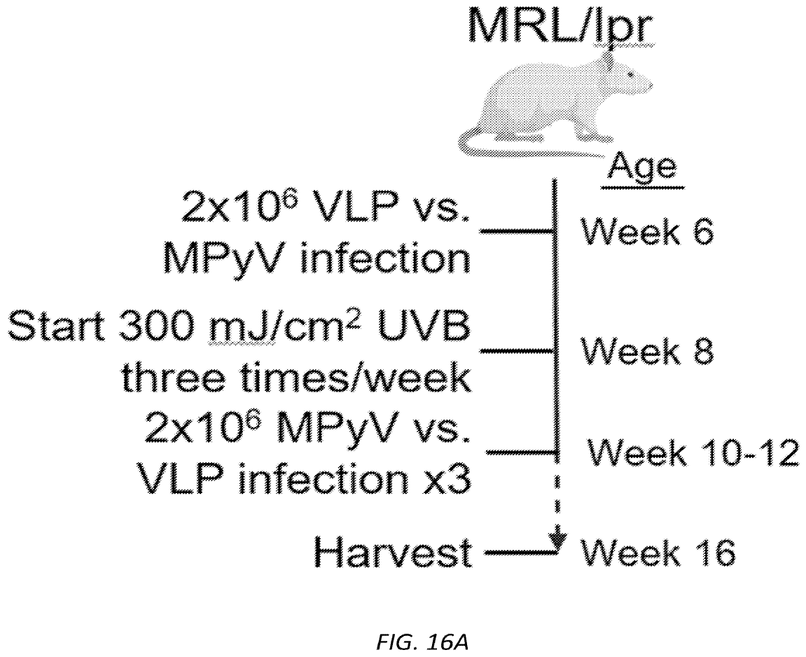

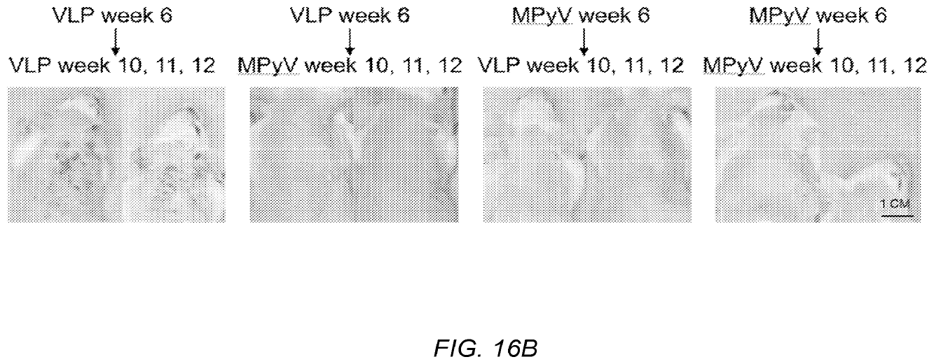

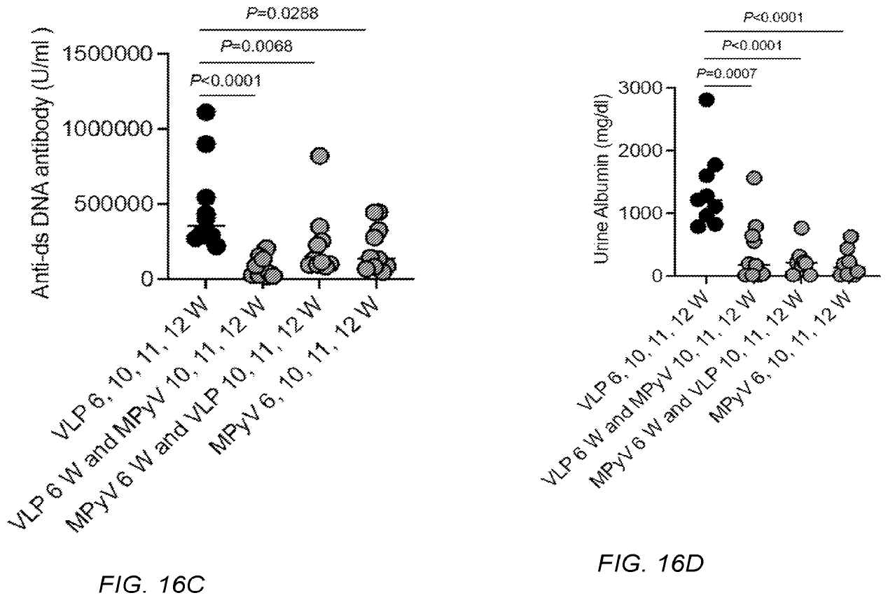

Six-week-old MRL/lpr mice received a dose of 2×10⁶ plaque-forming unit (PFU) live mouse polyomavirus (MPyV) versus equivalent virus-like particle (VLP) control via intradermal injection. Two weeks later, the mice were subjected to 300 mJ/cm² ultraviolet B (UVB) radiation, administered thrice weekly until they reached 16 weeks of age. At weeks 10, 11, and 12, mice received intradermal injections of MPyV versus VLP. 16-week-old mice were harvested to determine the extent of lupus phenotype in the animals (FIG. 16A). MPyV significantly reduced MRL/lpr mice skin dermatitis (FIG. 16B). Moreover, virus colonization before UVB treatment and virus therapy during UVB treatment led to a significant reduction in serum anti-dsDNA antibody levels (FIG. 16C) and a decrease in urine albumin concentration (FIG. 16D). These findings demonstrate that polyomavirus can be used as a therapeutic agent to treat lupus.

### REFERENCES

1. Moulton, V. R., et al., Pathogenesis of Human Systemic Lupus Erythematosus: A Cellular Perspective. Trends Mol Med, 2017. 23(7): p. 615-635.
2. Draborg, A. H., K. Duus, and G. Houen, Epstein-Barr virus and systemic lupus erythematosus. Clin Dev Immunol, 2012. 2012: p. 370516.
3. Jung, J. Y. and C. H. Suh, Infection in systemic lupus erythematosus, similarities, and differences with lupus flare. Korean J Intern Med, 2017. 32(3): p. 429-438.
4. Oldstone, M. B. and F. J. Dixon, Inhibition of antibodies to nuclear antigen and to DNA in New Zealand mice infected with lactate dehydrogenase virus. Science, 1972. 175(4023): p. 784-6.
5. Griffiths, P., Time to consider the concept of a commensal virus? Rev Med Virol, 1999. 9(2): p. 73-4.
6. Moustafa, A., et al., The blood DNA virome in 8,000 humans. PLoS Pathog, 2017. 13(3): p. e 1006292.
7. Ahsan, N. and K. V. Shah, Polyomaviruses and human diseases. Adv Exp Med Biol, 2006. 577: p. 1-18.
8. Kean, J. M., et al., Seroepidemiology of human polyomaviruses. PLoS Pathog, 2009. 5(3): p. e 1000363.
9. Sroller, V., et al., Seroprevalence rates of HPyV6, HPyV7, TSPyV, HPyV9, MWPyV and KIPyV polyomaviruses among the healthy blood donors. J Med Virol, 2016. 88(7): p. 1254-61.
10. Kamminga, S., et al., Prevalence of DNA of fourteen human polyomaviruses determined in blood donors. Transfusion, 2019. 59(12): p. 3689-3697.
11. Wieland, U., et al., Merkel cell polyomavirus DNA in persons without merkel cell carcinoma. Emerg Infect Dis, 2009. 15(9): p. 1496-8.
12. Foulongne, V., et al., Human skin microbiota: high diversity of DNA viruses identified on the human skin by high throughput sequencing. PLoS One, 2012. 7(6): p. e38499.
13. Foulongne, V., et al., Merkel cell polyomavirus DNA detection in lesional and nonlesional skin from patients with Merkel cell carcinoma or other skin diseases. Br J Dermatol, 2010. 162(1): p. 59-63.
14. Schowalter, R. M., et al., Merkel cell polyomavirus and two previously unknown polyomaviruses are chronically shed from human skin. Cell Host Microbe, 2010. 7(6): p. 509-15.
15. Schaffhausen, B. S., et al., Polyoma virus middle T antigen: relationship to cell membranes and apparent lack of ATP-binding activity. Mol Cell Biol, 1982. 2(10): p. 1187-98.
16. Dilworth, S. M., et al., Transformation by polyoma virus middle T-antigen involves the binding and tyrosine phosphorylation of Shc. Nature, 1994. 367(6458): p. 87-90.
17. Markland, W., et al., Site-directed mutagenesis of polyomavirus middle-T antigen sequences encoding tyrosine 315 and tyrosine 250. J Virol, 1986. 59(2): p. 384-91.
18. Dahl, J., et al., Evidence of a role for phosphatidylinositol 3-kinase activation in the blocking of apoptosis by polyomavirus middle T antigen. J Virol, 1998. 72(4): p. 3221-6.
19. Apostolidis, S. A., et al., The dysregulation of cytokine networks in systemic lupus erythematosus. J Interferon Cytokine Res, 2011. 31(10): p. 769-79.
20. Banchereau, J. and V. Pascual, Type I interferon in systemic lupus erythematosus and other autoimmune diseases. Immunity, 2006. 25(3): p. 383-92.
21. Ronnblom, L. and D. Leonard, Interferon pathway in SLE: one key to unlocking the mystery of the disease. Lupus Sci Med, 2019. 6(1): p. e 000270.
22. Liu, W., et al., Selective reactivation of STING signaling to target Merkel cell carcinoma. Proc Natl Acad Sci U S A, 2020. 117(24): p. 13730-13739.
23. Reus, J. B., et al., SV40 Large T Antigen Is Not Responsible for the Loss of STING in 293T Cells but Can Inhibit cGAS-STING Interferon Induction. Viruses, 2020. 12(2).
24. Hashida, Y., et al., Prevalence and Viral Loads of Cutaneous Human Polyomaviruses in the Skin of Patients With Chronic Inflammatory Skin Diseases. J Infect Dis, 2019. 219(10): p. 1564-1573.
25. Hooks, J. J., et al., Immune interferon in the circulation of patients with autoimmune disease. N Engl J Med, 1979. 301(1): p. 5-8.
26. Miller, J. C., et al., A critical role for type I IFN in arthritis development following Borrelia burgdorferi infection of mice. J Immunol, 2008. 181(12): p. 8492-503.
27. Elkon, K. B. and V. V. Stone, Type I interferon and systemic lupus erythematosus. J Interferon Cytokine Res, 2011. 31(11): p. 803-12.
28. Crow, Y. J. and N. Manel, Aicardi-Goutieres syndrome and the type I interferonopathies. Nat Rev Immunol, 2015. 15(7): p. 429-40.
29. Ladislau, L., et al., JAK inhibitor improves type I interferon induced damage: proof of concept in dermatomyositis. Brain, 2018. 141(6): p. 1609-1621.
30. Yao, Y., et al., Type I interferons in Sjogren's syndrome. Autoimmun Rev, 2013. 12(5): p. 558-66.
31. Guo, X., et al., Suppression of T Cell Activation and Collagen Accumulation by an Anti-IFNAR1 mAb, Anifrolumab, in Adult Patients with Systemic Sclerosis. J Invest Dermatol, 2015. 135(10): p. 2402-2409.
32. Davidson, S., et al., Pathogenic potential of interferon alphabeta in acute influenza infection. Nat Commun, 2014. 5: p. 3864.
33. Domizio, J. D., et al., The cGAS-STING pathway drives type I IFN immunopathology in COVID-19. Nature, 2022.
34. Henry, T., et al., Type I IFN signaling constrains IL-17A/F secretion by gammadelta T cells during bacterial infections. J Immunol, 2010. 184(7): p. 3755-67.
35. Auerbuch, V., et al., Mice lacking the type I interferon receptor are resistant to Listeria monocytogenes. J Exp Med, 2004. 200(4): p. 527-33.
36. Manca, C., et al., Hypervirulent M. tuberculosis W/Beijing strains upregulate type I IFNs and increase expression of negative regulators of the Jak-Stat pathway. J Interferon Cytokine Res, 2005. 25(11): p. 694-701.
37. Karaghiosoff, M., et al., Central role for type I interferons and Tyk2 in lipopolysaccharide-induced endotoxin shock. Nat Immunol, 2003. 4(5): p. 471-7.
38. Weighardt, H., et al., Type I IFN modulates host defense and late hyperinflammation in septic peritonitis. J Immunol, 2006. 177(8): p. 5623-30.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a condition associated with type 1 interferon overexpression in a patient comprising administering a therapeutically effective amount of a pharmaceutical composition comprising a polyomavirus to the patient.

2. The method of claim 1 wherein the polyomavirus is a live polyomavirus.

3. The method of claim 2 wherein the polyomavirus is a live attenuated polyomavirus.

4. The method of claim 2 or 3 wherein the polyomavirus is selected from HPyV6 and HPyV7.

5. The method of any one of the foregoing claims wherein the polyomavirus is administered by intradermal injection.

6. The method of any one of the foregoing claims wherein the condition is lupus.

7. A method for treating systemic lupus erythematosus, rheumatoid arthritis, Aicardi-Goutières syndrome, Dermatomyositis, systemic sclerosis, or Sjogren's syndrome, in a subject, the method comprising administering a therapeutically effective amount of live or live-attenuated polyomavirus, or an antigenic protein, protein fragment, or peptide thereof, to a subject identified as having lupus.

8. A method of vaccinating a subject against systemic lupus erythematosus, rheumatoid arthritis, Aicardi-Goutières syndrome, Dermatomyositis, systemic sclerosis, or Sjogren's syndrome, or reducing the risk of developing systemic lupus erythematosus, rheumatoid arthritis, Aicardi-Goutières syndrome, Dermatomyositis, systemic sclerosis, or Sjogren's syndrome, in a subject, the method comprising administering a therapeutically effective amount of live or live-attenuated polyomavirus, or antigenic protein, protein fragment, or peptide thereof, to the subject in need thereof.

9. The method of any of the above claims, wherein the live or live-attenuated polyomavirus, or antigenic protein, protein fragment, or peptide thereof is selected from MPyV, HPyV6 or HPyV7.

10. The method of any of the above claims, wherein the polyomavirus is a live polyomavirus.

11. The method of any of the above claims, wherein the polyomavirus is a live-attenuated polyomavirus.

12. The method of any of the above claims, wherein the polyomavirus is delivered using a vector.

13. The method of any of the above claims, wherein the vector is a viral vector.

14. The method of any of the above claims, wherein the viral vector is an adeno-associated virus (AAV) vector.

15. The method of any of the above claims, wherein the subject is a mammal.

16. The method of any of the above claims, wherein the subject is a human.

17. The method of any of the above claims, wherein the method is used to treat systemic lupus erythematosus (SLE).

18. The method of any of the above claims, wherein the live or live-attenuated polyomavirus, or an antigenic protein, protein fragment, or peptide thereof are administered systemically.

19. The method of any of the above claims, wherein the systemic administration comprises intradermal, subcutaneous, or intraperitoneal injections.

20. The method of any of the above claims, wherein treatment is monitored by observed dermatitis, size of spleen and/or lymph nodes, lupus nephritis, anti-DS DNA antibodies in the serum, urine protein concentration, or inflammatory cytokines in the skin.

21. The method of any of the above claims, further comprising administering to the subject an additional therapy for lupus.
